(19) United States
(12) Patent Application Publication
(10) Pub. No.: US 2023/0111265 A1
(43) Pub. Date: Apr. 13, 2023

(54) TREATMENT TOOL, TREATMENT SYSTEM, AND TREATMENT METHOD

(71) Applicant: OLYMPUS CORPORATION, Tokyo (JP)

(72) Inventors: Shinichi KITADE, Tokyo (JP); Kazuya NAKABE, Tokyo (JP)

(73) Assignee: OLYMPUS CORPORATION, Tokyo (JP)

(21) Appl. No.: 17/960,405

(22) Filed: Oct. 5, 2022

Related U.S. Application Data
(60) Provisional application No. 63/253,575, filed on Oct. 8, 2021.

(51) Int. Cl. A61B 18/14; A61B 17/32

(52) U.S. Cl. CPC A61B 18/1482; A61B 17/32002; A61B 2018/00601; A61B 2018/1412; A61B 2218/007

(57) ABSTRACT

A treatment tool includes a tubular member including an opening and a tubular member main body that has a tubular shape. The tubular member is configured to suction a tissue piece through the opening. The treatment tool includes a blade member provided on an opening edge of the opening.

### RELATED APPLICATION DATA

This application is based on and claims priority under 37 U.S.C. §119 to U.S. Provisional Application No. 63/253,575 filed on Oct. 8, 2021, the entire contents of which are incorporated herein by reference.

### FIELD OF THE DISCLOSURE

The present disclosure relates to a treatment tool, a treatment system, and a treatment method.

### BACKGROUND

A treatment tool called a morcellator has been used in enucleated prostate piece removal for removing prostate tissue (hereinbelow, referred to as the tissue piece) enucleated in holmium laser nucleation of the prostate (HoLEP), which is an operative treatment of Benign Prostatic Hyperplasia (BPH) (e.g., refer to Patent Literature 1).

A treatment tool disclosed in Patent Literature 1 includes an insertion portion including an inner tube having a first opening in a side wall and an outer tube having a second opening in a side wall, the inner tube being inserted inside the outer tube. The treatment tool sequentially cuts a tissue piece enucleated by laser radiation in holmium laser enucleation of the prostate and suctions the tissue piece through the inside of the inner tube, thereby discharging the tissue piece to the outside of the body from the inside of the bladder as described below.

Specifically, the inner tube and the outer tube are slid relative to each other along the longitudinal axis to allow the first and second openings to overlap each other. Since the suction is performed through the inner tube, a part of the tissue piece is drawn into the inner tube through an opening where the first and second openings overlap each other. Next, the inner tube and the outer tube are slid relative to each other along the longitudinal axis to narrow the area of the opening where the first and second openings overlap each other. Accordingly, the part of the tissue piece drawn into the inner tube through the opening is cut by an edge portion of the opening serving as a cutting blade. Further, the cut part of the tissue piece is discharged to the outside of the body through the inside of the inner tube.

The tissue piece is sequentially cut and discharged to the outside of the body from the inside of the bladder by repeatedly performing the above operations.

Patent Literature 1: US 6,024,751

### BRIEF SUMMARY OF EMBODIMENTS

A treatment tool includes a tubular member including an opening and a tubular member main body that has a tubular shape. The tubular member is configured to suction a tissue piece through the opening. The treatment tool includes a blade member provided on an opening edge of the opening.

### BRIEF DESCRIPTION OF THE DRAWINGS

FIG. 1 is a diagram illustrating a treatment system according to an embodiment.

FIG. 2 is a diagram describing the configuration of an insertion portion.

FIG. 3 is a diagram describing the configuration of the insertion portion.

FIG. 4 is a diagram describing the configuration of the insertion portion.

FIG. 5 is a diagram describing a treatment method.

FIG. 6 is a diagram describing the treatment method.

FIG. 7 is a diagram describing a first modification of the embodiment.

FIG. 8 is a diagram describing a second modification of the embodiment.

FIG. 9 is a diagram describing a third modification of the embodiment.

FIG. 10 is a diagram describing a fourth modification of the embodiment.

FIG. 11 is a diagram describing a fifth modification of the embodiment.

FIG. 12 is a diagram describing the fifth modification of the embodiment.

FIG. 13 is a diagram describing a sixth modification of the embodiment.

FIG. 14 is a diagram describing a seventh modification of the embodiment.

FIG. 15 is a diagram describing an eighth modification of the embodiment.

FIG. 16 is a diagram describing a ninth modification of the embodiment.

FIG. 17 is a diagram describing a tenth modification of the embodiment.

FIG. 18 is a diagram describing an eleventh modification of the embodiment.

FIG. 19 is a diagram describing the eleventh modification of the embodiment.

FIG. 20 is a diagram describing a twelfth modification of the embodiment.

FIG. 21 is a diagram describing a thirteenth modification of the embodiment.

FIG. 22 is a diagram describing a fourteenth modification of the embodiment.

FIG. 23 is a diagram describing a fifteenth modification of the embodiment.

### DETAILED DESCRIPTION

Hereinbelow, a mode for carrying out the present disclosure (hereinbelow, the embodiment) will be described with reference to the drawings. Note that the present disclosure is not limited to the embodiment described below. Furthermore, the same reference signs designate the same parts throughout the drawings.

### Configuration of Treatment System

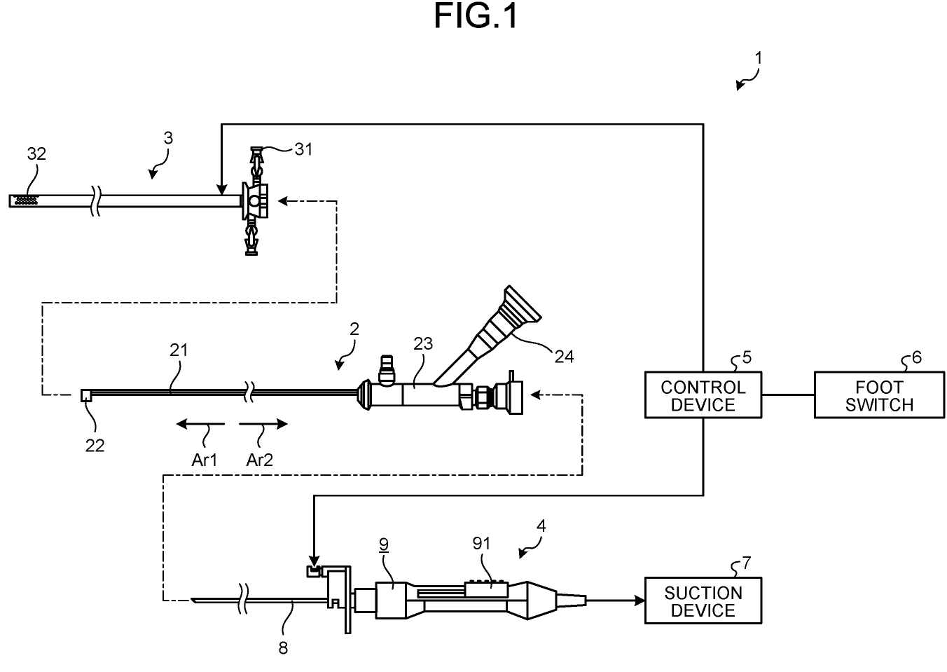

FIG. 1 is a diagram illustrating a treatment system 1 according to the embodiment. Note that, in FIG. 1, a dot-dash line arrow indicates an insertion direction, and a solid line arrow indicates control of each configuration or suction.

The treatment system 1 is an enucleated piece collecting system used in enucleated prostate piece removal. For example, the treatment system 1 is used in enucleated prostate piece removal for removing prostate tissue (hereinbelow, referred to as the tissue piece) enucleated in, for example, holmium laser enucleation of the prostate (HoLEP), which is an operative treatment of Benign Prostatic Hyperplasia (BPH). As illustrated in FIG. 1, the treatment system 1 includes an endoscope 2, an outer sheath 3, a treatment tool 4, a control device 5, a foot switch 6, and a suction device 7.

The endoscope 2 includes a rigid endoscope and is used to observe the inside of the bladder of a patient as a subject. As illustrated in FIG. 1, the endoscope 2 includes an endoscope main body 21, a distal end portion 22, a treatment tool insertion portion 23, and an eyepiece portion 24.

The endoscope main body 21 has a linearly extending elongated shape. Inside the endoscope main body 21, an optical system (not illustrated) that includes one or more lenses and collects light from a subject is provided.

In the following description, one side (the left side in FIG. 1) of the longitudinal axis of the endoscope main body 21 is referred to as a distal end side Ar1 (FIG. 1), and the other side (the right side in FIG. 1) is referred to as a proximal end side Ar2 (FIG. 1).

The distal end portion 22 is provided on the outer peripheral face of an end part of the endoscope main body 21 on the distal end side Ar1 and has a columnar shape that is eccentric from the central axis of the endoscope main body 21 and extends along the longitudinal axis of the endoscope main body 21. Although not specifically illustrated, the distal end portion 22 has a first through hole that penetrates the distal end portion 22 along the central axis of the column from the proximal end to the distal end and has a circular cross-sectional shape.

The treatment tool insertion portion 23 is provided on an end part of the endoscope main body 21 on the proximal end side Ar2 and has an elongated shape extending along the longitudinal axis of the endoscope main body 21. Although not specifically illustrated, the treatment tool insertion portion 23 has a second through hole that penetrates the treatment tool insertion portion 23 along the longitudinal axis from the proximal end to the distal end, is coaxial with the first through hole provided in the distal end portion 22, and has substantially the same inner diameter as the first through hole and has a circular cross-sectional shape.

The eyepiece portion 24 is connected to the proximal end of the endoscope main body 21 through the treatment tool insertion portion 23, and a camera head (not illustrated) is connected to the eyepiece portion 24. That is, a subject image that has been focused in the endoscope main body 21 and passed through the eyepiece portion 24 is captured by the camera head. Then, the endoscope image captured by the camera head is displayed on a display device (not illustrated).

The outer sheath 3 is at least partly made of a conductive material and has a cylindrical shape having an inner diameter slightly larger than the outer diameter of the distal end portion 22. As indicated by the dot-dash line arrow in FIG. 1, the distal end portion 22 and the endoscope main body 21 are inserted inside the outer sheath 3. Accordingly, an open part of the outer sheath 3 on the distal end side Ar1 is closed by the distal end portion 22. On the other hand, an open part of the outer sheath 3 on the proximal end side Ar2 is closed by the treatment tool insertion portion 23.

The outer sheath 3 is provided with, on an end part on the proximal end side Ar2, a water feed port 31 (FIG. 1) to which a tube (not illustrated) is connected for feeding a physiological saline solution to the inside of the outer sheath 3.

The outer sheath 3 also has, in a side wall of an end part on the distal end side Ar1, a water feed hole 32 (FIG. 1) that allows communication between the inside and outside of the outer sheath 3.

That is, a physiological saline solution fed to the inside of the outer sheath 3 through the water feed port 31 flows out of the outer sheath 3 through the water feed hole 32.

The treatment tool 4 is a so-called morcellator that suctions a tissue piece enucleated in enucleation of the prostate to discharge the tissue piece to the outside of the body from the inside of the bladder. As illustrated in FIG. 1, the treatment tool 4 includes an insertion portion 8 and a housing 9.

The insertion portion 8 corresponds to the tubular member according to the present disclosure. The insertion portion 8 is formed in a linearly extending elongated shape and has an outer diameter slightly smaller than the inner diameter of the first and second through holes described above. As indicated by the dot-dash line arrow in FIG. 1, the insertion portion 8 is inserted into the first and second through holes described above. Accordingly, the distal end part of the insertion portion 8 projects toward the distal end side Ar1 from the distal end (the distal end portion 22) of the outer sheath 3 inserted inside the bladder.

The detailed configuration of the insertion portion 8 will be described further below.

The housing 9 is a portion that is provided on an end part of the insertion portion 8 on the proximal end side Ar2 and grasped by an operator, such as a surgeon.

The housing 9 is provided with an operating unit 91 that receives a suction start operation and a suction finish operation as user operations performed by an operator, such as a surgeon. The operating unit 91 is movable along the longitudinal axis of the insertion portion 8. Here, the suction start operation is, for example, an operation of moving the operating unit 91 toward the distal end side Ar1. When the suction start operation is performed, suction of a tissue piece is started. On the other hand, the suction finish operation is, for example, an operation of moving the operating unit 91 toward the proximal end side Ar2. When the suction finish operation is performed, the suction of the tissue piece is finished.

Note that the suction start operation and the suction finish operation are not limited to the operations described above and may be an operation using a push button switch or an operation using a lever.

The control device 5 supplies high frequency power between the outer sheath 3 and a sheath 81 (described further below) constituting the insertion portion 8 in response to the treatment start operation as a user operation of an operator, such as a surgeon, on the foot switch 6.

Note that the treatment start operation is not limited to the user operation on the foot switch 6 and may be an operation using a switch operated with a hand.

The suction device 7 includes a tank, a pump, and the like, and suctions and collects a tissue piece through the treatment tool 4.

### Configuration of Insertion Portion

Next, the configuration of the above-described insertion portion 8 will be described.

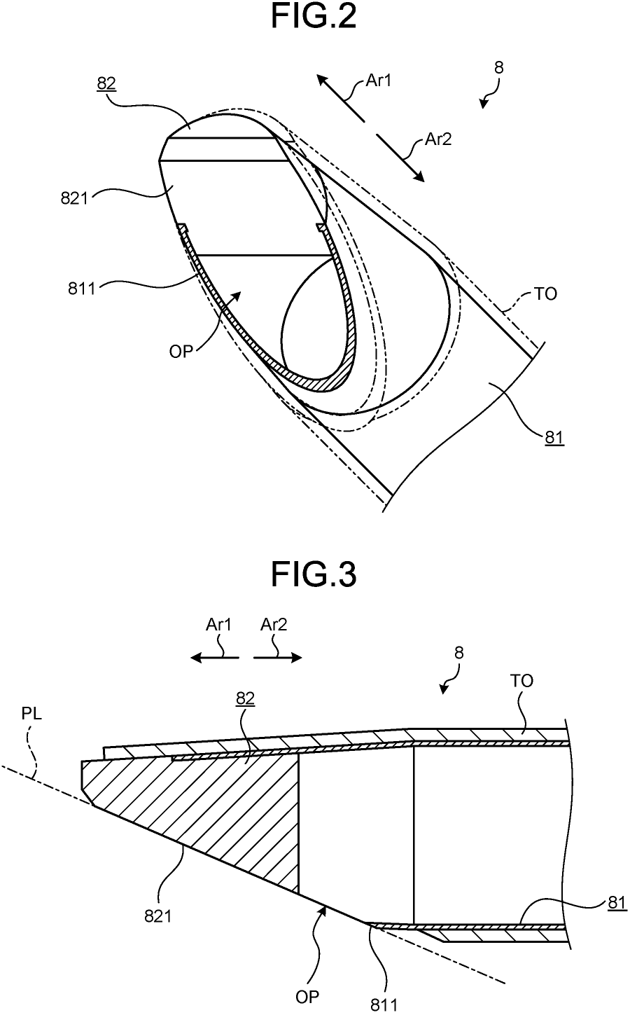

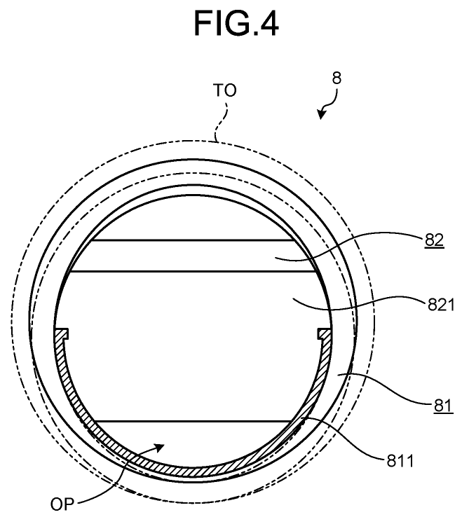

FIGS. 2 to 4 are diagrams describing the configuration of the insertion portion 8. Specifically, FIG. 2 is a perspective view of an end part of the insertion portion 8 on the distal end side Ar1. FIG. 3 is a sectional view of the end part of the insertion portion 8 on the distal end side Ar1 taken along a plane extending along the longitudinal axis of the insertion portion 8. FIG. 4 is a diagram illustrating the distal end of the insertion portion 8 viewed along the longitudinal axis of the insertion portion 8. Note that, in FIGS. 2 and 4, for convenience of description, an outer tube TO is indicated by a chain double-dashed line. Also, in FIGS. 2 and 4, for convenience of description, an electrode 811 is hatched.

As illustrated in FIGS. 2 to 4, the insertion portion 8 includes the sheath 81 and a non-blade member 82.

The sheath 81 corresponds to the tubular member main body according to the present disclosure. The sheath 81 is a cylindrical pipe made of a conductive material and serves as a pipe line. An end part of the sheath 81 on the proximal end side Ar2 is supported by the housing 9. The suction device 7 suctions a tissue piece through the sheath 81.

In the sheath 81, as illustrated in FIGS. 2 or 3, the end part on the distal end side Ar1 has a tapered shape whose diameter decreases toward the distal end side Ar1.

Also, in the sheath 81, an end face on the distal end side Ar1 has a shape cut on a plane PL (FIG. 3) intersecting the longitudinal axis of the sheath 81.

The non-blade member 82 is made of, for example, rubber having an electrical insulating property and elasticity. The non-blade member 82 is attached to the distal end of the sheath 81 by insert molding or the like and closes a part of an open part of the sheath 81 on the distal end side Ar1. The remaining part of the open part of the sheath 81 on the distal end side Ar1 that is not closed by the non-blade member 82 corresponds to an opening OP (FIGS. 2 to 4) according to the present disclosure. That is, the opening OP is defined by the sheath 81 and the non-blade member 82. In the present embodiment, the opening OP is located on an imaginary plane inclined from the distal end side Ar1 toward the proximal end side Ar2 in the tubular member 8.

Note that the non-blade member 82 is not limited to rubber and may be made of a material having an electrical insulating property such as ceramic or resin.

In the present embodiment, the non-blade member 82 is attached to the distal end of the sheath 81 in such a state that the opening OP is located on the proximal end side Ar2 relative to the non-blade member 82. That is, the non-blade member 82 is also a distal end member. An end of the sheath 81 on the distal end side Ar1 is in a state where a portion on the distal end side Ar1 is covered by the non-blade member 82 and only a U-shaped portion on the proximal end side Ar2 (electrode 811) is exposed. That is, the electrode 811 is provided on a suction direction side (the proximal end side Ar2) of the opening edge of the opening OP where a tissue piece is suctioned into the sheath 81 through the opening OP. More specifically, a portion of the opening edge of the opening OP located on the most proximal end side Ar2 corresponds to a valley portion of the U shape of the electrode 811. The distal end side Ar1 of the opening edge of the opening OP is constituted of the non-blade member 82 and has a linear shape perpendicular to the longitudinal axis of the sheath 81. That is, the non-blade member 82 and the electrode 811 that constitute the opening edge of the opening OP have a positional relationship in which the electrode 811 is located on the suction direction side (the proximal end side Ar2) relative to the non-blade member 82.

Note that, in the present embodiment, both ends of the U shape of the electrode 811 extend up to a portion of the opening OP other than the opening edge as illustrated in FIGS. 2 or 4.

As illustrated in FIGS. 2 to 4, the non-blade member 82 is provided with a sliding surface 821 that allows a tissue piece to slide thereon toward the opening OP when the tissue piece is suctioned into the sheath 81 through the opening OP. In the present embodiment, an opening plane of the opening OP and the sliding surface 821 are located on the same plane PL to form a contiguous plane with no step (FIG. 3). That is, the opening plane of the opening OP intersects the longitudinal axis of the sheath 81. In other words, the sliding surface 821 is inclined from the distal end side Ar1 toward the proximal end side Ar2. A ridge line of the sliding surface 821 on the proximal end side Ar2 forms a part of the opening edge.

As illustrated in FIGS. 2 to 4, the outer face of the sheath 81 and the outer face of the non-blade member 82 are covered by the outer tube TO having an electrical insulating property and flexibility with the electrode 811 and the sliding surface 821 exposed to the outside. Here, the electrode 811 projects toward the distal end side Ar1 relative to the outer tube TO.

### Treatment Method

Next, a treatment method using the above-described treatment system 1 will be described. Note that, in the following, for convenience of description, a method of discharging, from the inside of the bladder to the outside of the body, a tissue piece enucleated in enucleation of the prostate using the treatment tool 4 will be mainly described.

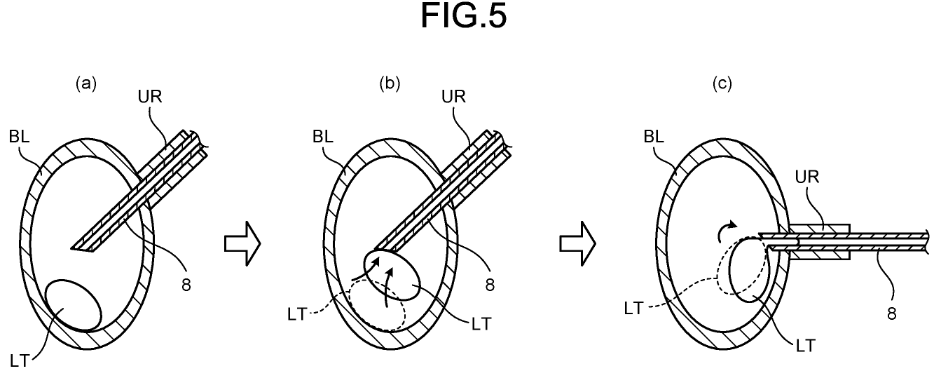

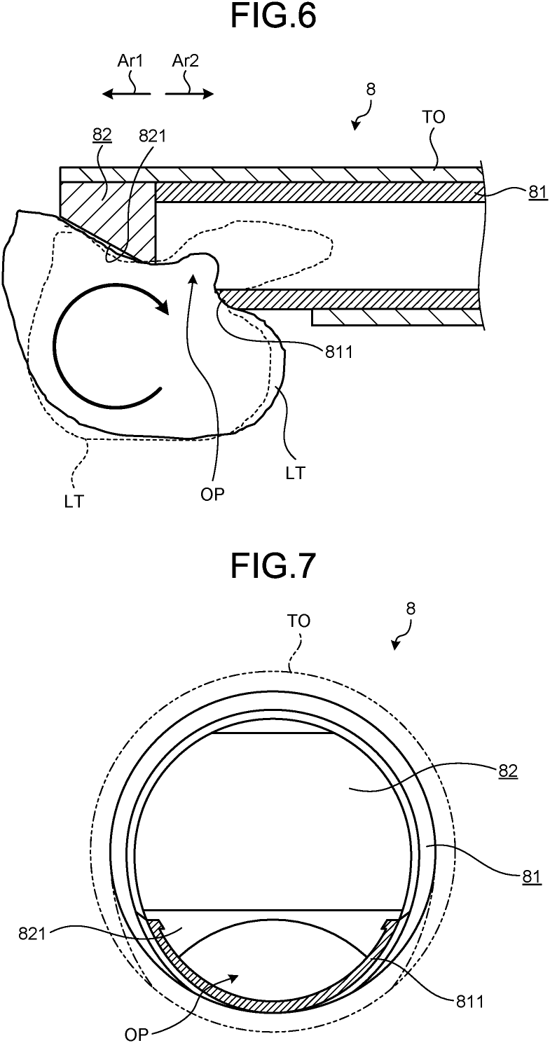

FIGS. 5 and 6 are diagrams describing the treatment method. FIG. 5 illustrates a state in which the insertion portion 8 is inserted inside the bladder BL through the urethra UR. FIG. 6 is an enlarged view of (c) of FIG. 5.

While, in actuality, the outer sheath 3 and the endoscope main body 21 are also inserted into the bladder BL through the urethra UR together with the insertion portion 8, illustration of the outer sheath 3 and the like is omitted in FIG. 5 for convenience of description. The inside of the bladder BL is filled with a physiological saline solution flowing through a path passing the water feed port 31, the inside of the outer sheath 3, and the water feed hole 32 in this order.

First, an operator, such as a surgeon, performs the suction start operation on the operating unit 91. Accordingly, the physiological saline solution is suctioned into the sheath 81 through the opening OP. Then, as illustrated in (a) of FIG. 5 and (b) of FIG. 5, inside the bladder BL, an enucleated tissue piece LT is attracted to the distal end of the insertion portion 8 and held on the distal end of the insertion portion 8 (the sliding surface 821 and the opening OP) .

Next, the operator, such as a surgeon, performs a treatment start operation on the foot switch 6. Accordingly, the control device 5 supplies high frequency power between the outer sheath 3 and the sheath 81. At this time, the outer sheath 3 functions as a collection electrode. On the other hand, the electrode 811 functions as an active electrode and scrapes off a part of the tissue piece LT in response to the supplied high frequency power on a principle described further below. Then, the tissue piece LT is gradually scraped off by the electrode 811 while being turned in the direction of an arrow indicated in (c) of FIG. 5 or FIG. 6, and the scraped-off part of the tissue piece LT is suctioned into the sheath 81 through the opening OP. That is, after a part of the tissue piece LT is suctioned, the suction is continued to turn the tissue piece LT, the electrode 811 scrapes off another part of the tissue piece, and another scraped-off part of the tissue piece LT is further suctioned through the opening OP. Note that the tissue piece LT scraped off by the electrode 811 is suctioned into the sheath 81 not in a finely tone state, but in a connected state. A broken line of FIG. 6 indicates a state in which the tissue piece LT scraped off by the electrode 811 is suctioned into the sheath 81 in a connected state.

That is, the treatment method according to the present embodiment continuously suctions the tissue piece LT into the sheath 81 through the opening OP without changing the opening area of the opening OP.

Note that the principle of cutting the tissue piece LT using the electrode 811 is as follows.
(1) When the supply of high frequency power between the outer sheath 3 and the sheath 81 is started, the physiological saline solution around the electrode 811 is heated by high frequency current from the electrode 811.
(2) When the physiological saline solution around the electrode 811 reaches the boiling point, the physiological saline solution evaporates, and air bubbles are thus generated. When the supply of high frequency power is further continued, more air bubbles are generated.
(3) When the air bubbles cover the electrode 811 and its vicinity, a high resistance state occurs between the entire electrode 811 and the physiological saline solution, which causes spark discharge. Heat of the spark discharge generates more air bubbles, and the spark discharge continues.
(4) When the electrode 811 in the spark discharge state in its entirety is brought into contact with the tissue piece LT, heat of the spark discharge heats the tissue piece LT and thus causes steam explosion, which scrapes the tissue piece LT.

As described above, the spark discharge described above is important for cutting the tissue piece LT by the electrode 811.

The present embodiment described above achieves the following effects.

In the treatment tool 4 according to the present embodiment, the electrode 811 functioning as the blade member that scrapes the tissue piece LT is provided only on a part of the opening edge of the opening OP. In addition, the non-blade member 82 is provided on at least a part of the opening edge outside the area in which the electrode 811 that functions as the blade member is provided. More specifically, the electrode 811 is provided on the suction direction side (the proximal end side Ar2) of the opening edge of the opening OP. Thus, it is possible to sequentially scrape off the tissue piece LT using the electrode 811 and discharge the tissue piece LT to the outside of the body by continuously rubbing the surface of tissue piece LT while turning the tissue piece LT ((c) of FIG. 5, FIG. 6) without moving the electrode 811.

That is, unlike the treatment tool disclosed in Patent Literature 1 in which the cutting blade moves, it is possible to scrape off and collect the tissue piece LT by not moving the electrode 811 serving as the cutting blade, but moving the tissue piece LT in the present embodiment. The tissue piece LT can be turned relative to the treatment tool 4 because the tissue piece LT can move freely inside the bladder BL. On the other hand, the bladder wall does not move freely. Thus, for example, even when the distal end part of the insertion portion 8 makes contact with the bladder wall, the electrode 811 does not dig into the bladder wall more than a certain depth. Thus, it is possible to prevent an unintended effect on living tissue. Also, even when the bladder wall bleeds, the bleeding can be stopped by pressing the electrode 811 against the bleeding bladder wall using a known hemostasis mode by the control device 5.

Since the tissue piece LT is suctioned not after cut into small pieces, but sequentially scraped off and suctioned while being turned, the efficiency of collecting the tissue piece LT can be improved.

Thus, the treatment tool 4 according to the present embodiment can improve the efficiency of collecting the tissue piece LT while preventing an unintended effect on living tissue.

In particular, the blade member according to the present disclosure is the electrode 811 that scrapes the tissue piece LT in response to high frequency power supplied.

Thus, for example, the electrode 811 can also easily burn off hard tissue that may induce the Beach Balls (BB) phenomenon, and the efficiency of collecting the tissue piece LT can be further improved.

In the treatment tool 4 according to the present embodiment, the non-blade member 82 constituting a part of the opening edge of the opening OP other than the electrode 811 has the sliding surface 821 that allows the tissue piece LT to slide thereon toward the opening OP. The sliding surface 821 and the opening plane of the opening OP form the contiguous plane with no gap and are located on the plane PL intersecting the longitudinal axis of the sheath 81.

Thus, when the electrode 811 scrapes off the tissue piece LT, the tissue piece LT can be turned smoothly without caught on a step, and the efficiency of collecting the tissue piece LT can be even further improved. Furthermore, since a part of the non-blade member 82 made of a material having an electrical insulating property is continuously located on the surface of the tissue piece LT, the tissue piece LT is attracted to the opening OP without piercing. Thus, the tissue piece LT is scraped off while being turned in the opening OP.

In the treatment tool 4 according to the present embodiment, the non-blade member 82 is made of an elastic member and provided on the distal end of the sheath 81. That is, in the insertion portion 8, the distal end that is likely to come into contact with the bladder wall or the like is constituted of the elastic member.

Thus, even when the distal end of the insertion portion 8 makes contact with the bladder wall or the like, the bladder wall or the like is not injured. Thus, it is possible to reliably prevent an unintended effect on living body.

In the treatment tool 4 according to the present embodiment, both the ends of the U shape of the electrode 811 extend up to the portion of the opening OP other than the opening edge. Also, the electrode 811 projects toward the distal end side Ar1 relative to the outer tube TO.

Thus, for example, when the electrode 811 scrapes off the tissue piece LT, even in a state where the tissue piece LT is held on the distal end of the insertion portion 8, both the ends of the U shape of the electrode 811 can be kept in contact with the physiological saline solution. Accordingly, it is possible to stabilize spark discharge, which is important for scraping off the tissue piece LT in the electrode 811, and efficiently scrape off the tissue piece LT. Furthermore, since the electrode 811 projects toward the distal end side Ar1 relative to the outer tube TO, the electrode 811 can be reliably brought into contact with the tissue piece LT.

In the treatment tool 4 according to the present embodiment, the end part of the sheath 81 on the distal end side Ar1 has the tapered shape whose diameter decreases toward the distal end side Ar1.

Thus, it is possible to prevent the tissue piece LT scraped off by the electrode 811 and suctioned into the sheath 81 through the opening OP from coming into contact with the inner face of the sheath 81 and smoothly suction the tissue piece LT.

Note that, while the end part of the sheath 81 on the distal end side Ar1 has the tapered shape in the present embodiment, the present disclosure is not limited thereto, and the end part of the sheath 81 on the distal end side Ar1 may have a shape extending with a constant diameter as a matter of course.

### Other Embodiments

While the mode for carrying out the present disclosure has been described above, the present disclosure should not be limited only to the embodiment described above.

While, in the embodiment described above, the electrode 811 is employed as the blade member according to the present disclosure, the present disclosure is not limited thereto. A blade or resin that performs mechanical scraping may be employed, or any member capable of scraping a tissue piece such as fiber laser may be employed. In this case, as the non-blade member 82, a member that can allow the tissue piece to slide thereon such as metal may be employed.

While, in the embodiment described above, the shape of the blade member according to the present disclosure is the U shape, the present disclosure is not limited thereto. Another shape such as a V shape or an I shape may be employed.

### First Modification

FIG. 7 is a diagram describing a first modification of the embodiment. Specifically, FIG. 7 corresponds to FIG. 4.

In the embodiment described above, the shape of the opening edge of the opening OP may be a shape in the first modification illustrated in FIG. 7.

Specifically, the distal end side Ar1 of the opening edge of the opening OP according to the first modification is constituted of the non-blade member 82 and has a curved shape convex away from the electrode 811 as illustrated in FIG. 7. The proximal end side Ar2 of the opening edge of the opening OP has a U shape constituted of the electrode 811 as with the embodiment described above.

The first modification described above achieves the following effect in addition to the same effects as described in the embodiment.

If the depth of the scraping performed by the electrode 811 increases, clogging is likely to occur inside the pipe line. In order to prevent this clogging, the opening OP according to the first modification is expanded so as to be convex away from the electrode 811, which makes it possible to make the opening area of the opening OP relatively large while maintaining the depth of cutting the tissue piece LT. Thus, it is possible to increase a force of suctioning the tissue piece LT through the opening OP and smoothly turn the tissue piece LT when the electrode 811 scrapes off the tissue piece LT. Thus, the efficiency of collecting the tissue piece LT can be even further improved.

Note that the shape of the distal end side Ar1 of the opening edge of the opening OP may be a curved shape convex toward the electrode 811.

### Second Modification

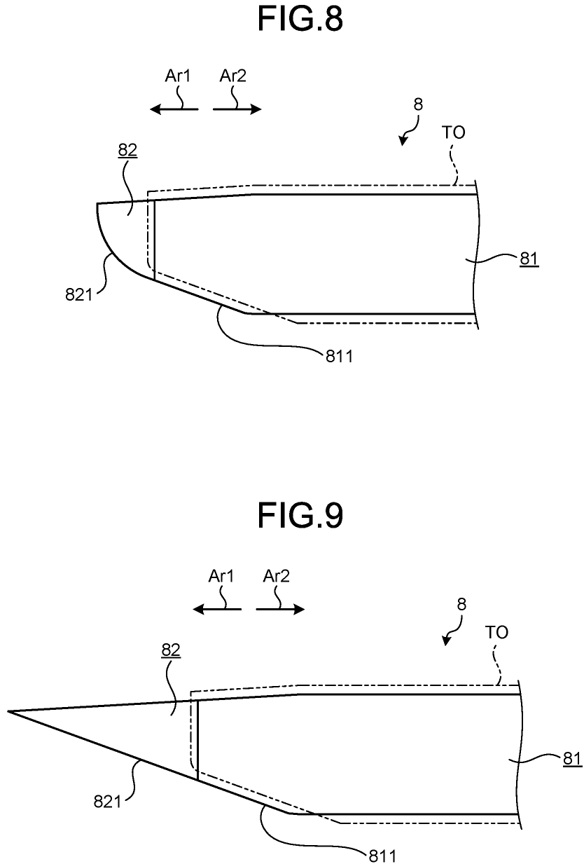

FIG. 8 is a diagram describing a second modification of the embodiment. Specifically, FIG. 8 is a diagram illustrating an end part of the insertion portion 8 on the distal end side Ar1 according to the second modification viewed in the direction perpendicular to the longitudinal axis of the insertion portion 8. Note that, for convenience of description, the outer tube TO is indicated by a chain double-dashed line in FIG. 8.

In the embodiment described above, the shape of the non-blade member 82 may be a shape in the second modification illustrated in FIG. 8.

Specifically, as illustrated in FIG. 8, an edge portion of the non-blade member 82 according to the second modification is round-chamfered. In the non-blade member 82 according to the second modification, the sliding surface 821 is a curved surface convex toward the distal end side Ar1. Note that, in the second modification, the opening plane of the opening OP and the sliding surface 821 form a contiguous plane with no step as with the embodiment described above.

The second modification described above achieves the following effects in addition to the same effects as described in the embodiment.

The edge portion of the non-blade member 82 according to the second modification is round-chamfered.

Thus, even when the distal end (the non-blade member 82) of the insertion portion 8 makes contact with the bladder wall of the like, the bladder wall or the like is not injured. In addition, even when the shape of the tissue piece LT has asperities, followability to the tissue piece LT is improved. Thus, it is possible to reliably prevent an unintended effect on living tissue.

Note that an edge portion of the opening edge of the opening OP may also be round-chamfered.

### Third Modification

FIG. 9 is a diagram describing a third modification of the embodiment. Specifically, FIG. 9 is a diagram illustrating an end part of the insertion portion 8 on the distal end side Ar1 according to the third modification viewed in the direction perpendicular to the longitudinal axis of the insertion portion 8. Note that, for convenience of description, the outer tube TO is indicated by a chain double-dashed line in FIG. 9.

In the embodiment described above, the shape of the non-blade member 82 may be a shape in the third modification illustrated in FIG. 9.

Specifically, as illustrated in FIG. 9, the non-blade member 82 according to the third modification has a shape pointed toward the distal end side Ar1. Even when the non-blade member 82 has the shape pointed toward the distal end side Ar1 in this manner, the non-blade member 82 is easily deformed due to its elasticity and does not pierce living tissue.

### Fourth Modification

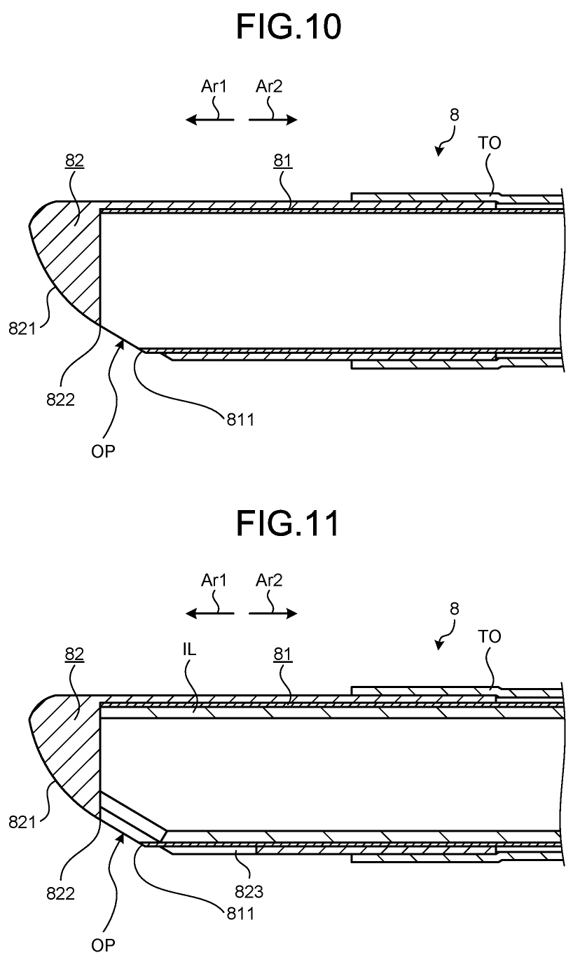

FIG. 10 is a diagram describing a fourth modification of the embodiment. Specifically, FIG. 10 corresponds to FIG. 3.

In the embodiment described above, the shape of the non-blade member 82 may be a shape in the fourth modification illustrated in FIG. 10.

Specifically, as illustrated in FIG. 10, the non-blade member 82 according to the fourth modification has a bottomed cylindrical shape inside which the end part of the sheath 81 on the distal end side Ar1 is inserted. The non-blade member 82 has, on a bottom portion thereof, a through hole 822 that penetrates the non-blade member 82 from the inside to outside thereof. The electrode 811 projects out of the non-blade member 82 toward the distal end side Ar1 through the through hole 822. An area surrounded by the through hole 822 and the electrode 811 functions as the opening OP. Also, the outer face of the bottom portion of the non-blade member 82 functions as the sliding surface 821.

The fourth modification described above achieves the following effect in addition to the same effects as described in the embodiment.

According to the non-blade member 82 according to the fourth modification, since the end part of the sheath 81 on the distal end side Ar1 can be covered by the non-blade member 82, a sufficient creepage distance between the physiological saline solution located outside and the sheath 81 can be maintained.

### Fifth Modification

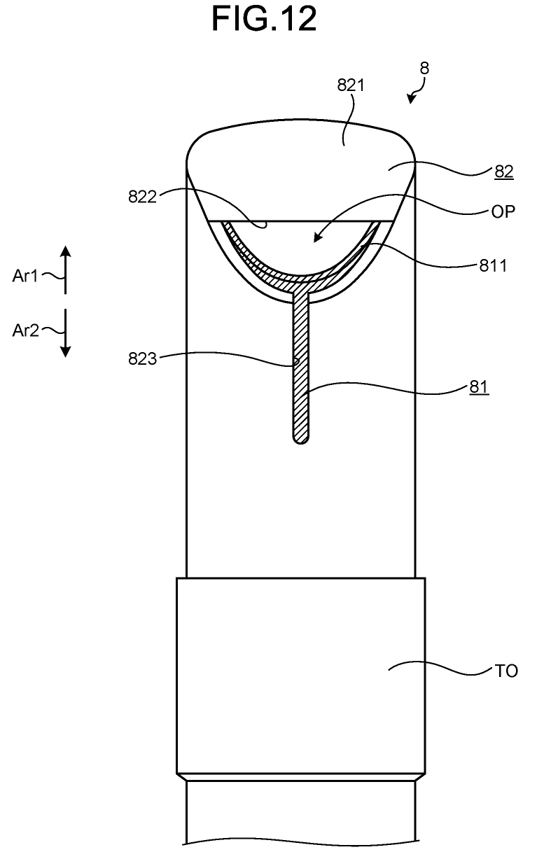

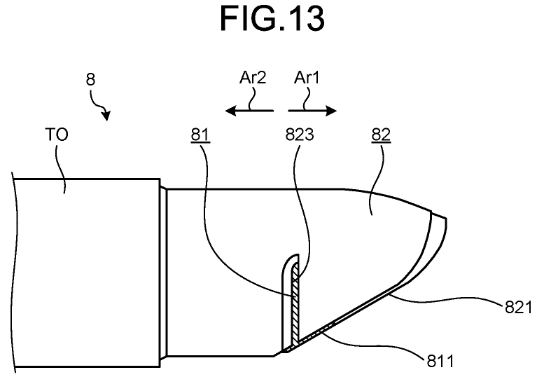

FIGS. 11 and 12 are diagrams describing a fifth modification of the embodiment. Specifically, FIG. 11 corresponds to FIG. 3. FIG. 12 is a diagram illustrating an end part of the insertion portion 8 on the distal end side Ar1 according to the fifth modification viewed from the lower side of FIG. 11. Note that, for convenience of description, the electrode 811 is hatched in FIG. 12.

In the fourth modification described above, an insulating layer IL (FIG. 11) may be provided on the inner face of the sheath 81, and the non-blade member 82 may be provided with a groove 823 as with the fifth modification illustrated in FIGS. 11 and 12.

The insulating layer IL is made of a material having an electrical insulating property and provided on the inner face of the sheath 81 as illustrated in FIG. 11. Note that the insulating layer IL can be, for example, coating.

As illustrated in FIGS. 11 or 12, the groove 823 penetrates the non-blade member 82 from the inside to outside thereof and linearly extends by a predetermined dimension toward the proximal end side Ar2 from a part of the inner peripheral edge of the through hole 822 located on the most proximal end side Ar2. A part of the outer face of the sheath 81 other than the electrode 811 is also exposed through the groove 823.

The fifth modification described above achieves the following effect in addition to the same effects as described in the embodiment and the fourth modification.

In the fifth modification, the insulating layer IL is provided on the inner face of the sheath 81.

Thus, the surface area of the sheath 81 that functions as the active electrode can be minimized. Accordingly, even when the inside of the sheath 81 is filled with the physiological saline solution, it is possible to stabilize spark discharge, which is important for scraping off the tissue piece LT in the electrode 811, and efficiently scrape off the tissue piece LT.

Also, in the fifth modification, the non-blade member 82 is provided with the groove 823 so that the electrode 811 extends.

Thus, for example, a state where, when the electrode 811 scrapes off the tissue piece LT, the tissue piece LT is held on the distal end of the insertion portion 8, and the electrode 811 is covered by the tissue piece LT is assumed. According to the fifth modification, even in such a state, a part of the outer face of the sheath 81 other than the electrode 811 is kept in contact with the physiological saline solution through the groove 823. Accordingly, it is possible to stabilize spark discharge, which is important for scarping off the tissue piece LT in the electrode 811, and efficiently scrape off the tissue piece LT.

### Sixth Modification

FIG. 13 is a diagram describing a sixth modification of the embodiment. Specifically, FIG. 13 is a diagram illustrating an end part of the insertion portion 8 on the distal end side Ar1 according to the sixth modification viewed in the direction perpendicular to the longitudinal axis of the insertion portion 8. Note that, for convenience of description, the electrode 811 is hatched in FIG. 13.

In the fifth modification described above, the position where the groove 823 is provided is not limited to the part of the inner peripheral edge of the through hole 822 located on the most proximal end side Ar2 and may be another position on the inner peripheral edge as with the sixth modification illustrated in FIG. 13.

Even when the position where the groove 823 is provided is changed as with the sixth modification described above, the same effects as described in the fifth modification are achieved. Note that the position, shape, number of grooves are not limited to these as long as the groove is connected to the electrode 811. For example, grooves 823 may be provided at two or more locations.

### Seventh Modification

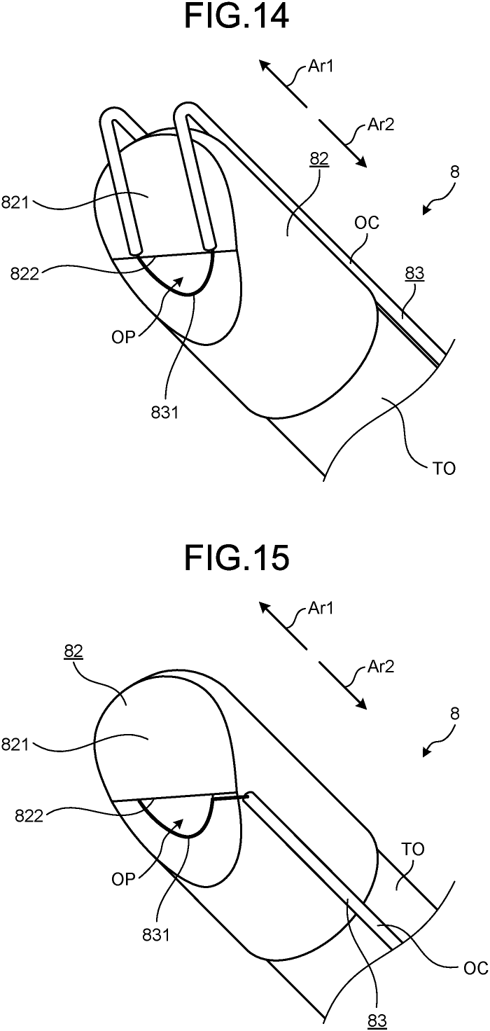

FIG. 14 is a diagram describing a seventh modification of the embodiment. Specifically, FIG. 14 is a perspective view of an end part of the insertion portion 8 on the distal end side Ar1 according to the seventh modification.

In the fifth modification described above, instead of the electrode 811, a conductor portion constituting a part of a lead wire 83 may serve as an electrode 831 as with the seventh modification illustrated in FIG. 14.

Specifically, the non-blade member 82 according to the seventh modification covers the entire end of the sheath 81 on the distal end side Ar1 as illustrated in FIG. 14. That is, in the seventh modification, the through hole 822 functions as the opening OP.

The lead wire 83 extends from the proximal end to the distal end of the insertion portion 8, and is routed along the U-shaped portion of the inner peripheral edge of the through hole 822 located on the proximal end side Ar2 and then routed from the distal end to the proximal end of the insertion portion 8. An outer jacket TI of the lead wire 83 is provided on a part of the lead wire 83 other than the electrode 831, which is the conductor portion routed along the U-shaped portion of the inner peripheral edge of the through hole 822.

In the seventh modification, the control device 5 supplies high frequency power between the outer sheath 3 and the lead wire 83 in response to the treatment start operation of an operator, such as a surgeon, on the foot switch 6. Accordingly, the electrode 831 functions in the same manner as the electrode 811 described in the above embodiment.

Even when the electrode 831 according to the seventh modification described above is employed, the same effects as described in the embodiment and the fifth modification are achieved.

### Eighth Modification

FIG. 15 is a diagram describing an eighth modification of the embodiment. Specifically, FIG. 15 is a perspective view of an end part of the insertion portion 8 on the distal end side Ar1 according to the eighth modification.

The seventh modification described above may employ a configuration in which one end portion of one lead wire 83 serves as the electrode 831, and the electrode 831 is deformed into a shape extending along the U-shaped portion of the inner peripheral edge of the through hole 822 located on the proximal end side Ar2 as with the eighth modification illustrated in FIG. 15.

Even when the electrode 831 according to the eighth modification described above is employed, the same effects as described in the embodiment and the fifth and seventh modifications are achieved.

### Ninth Modification

FIG. 16 is a diagram describing a ninth modification of the embodiment. Specifically, FIG. 16 is a perspective view of an end part of the insertion portion 8 on the distal end side Ar1 according to the ninth modification.

In the embodiment described above, the positional relationship between the electrode 811 and the non-blade member 82 constituting the opening edge of the opening OP may be a positional relationship in the ninth modification illustrated in FIG. 16.

Specifically, relative to the opening OP described above in the embodiment, the opening OP according to the ninth modification is turned around the central axis of the sheath 81 by an angle larger than 0° and smaller than 90°. Accordingly, the valley portion of the U shape of the electrode 811 is displaced from the portion of the opening edge of the opening OP located on the most proximal end side Ar2. That is, the non-blade member 82 and the electrode 811 constituting the opening edge of the opening OP have a positional relationship in which the non-blade member 82 and the electrode 811 are arrange in parallel in the direction perpendicular to the longitudinal axis of the sheath 81.

Even when the electrode 811 according to the ninth modification described above is employed, the same effects as described in the embodiment are achieved.

### Tenth Modification

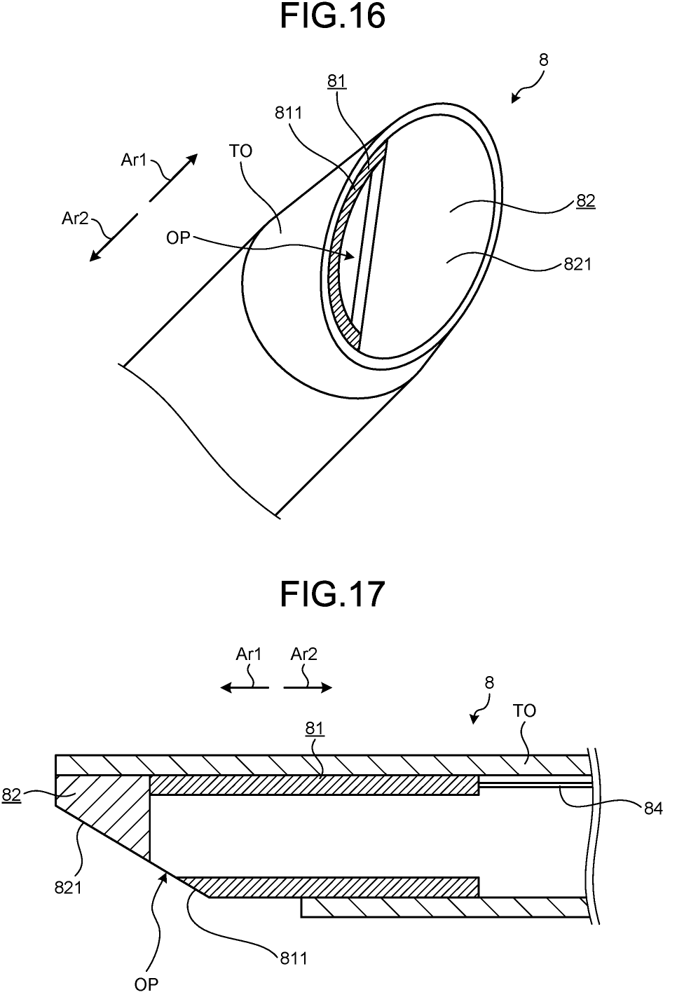

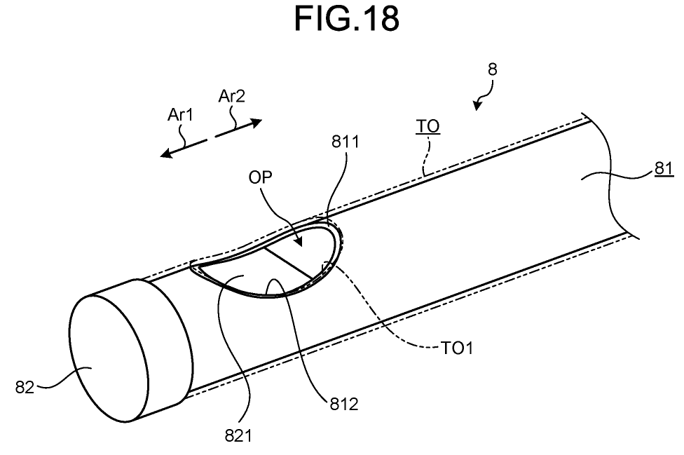

FIG. 17 is a diagram describing a tenth modification of the embodiment. Specifically, FIG. 17 corresponds to FIG. 3.

In the embodiment described above, the configuration of the insertion portion 8 may be a configuration in the tenth modification illustrated in FIG. 17.

Specifically, in the insertion portion 8 according to the tenth modification, the sheath 81 is provided only on the distal end part of the insertion portion 8 as illustrated in FIG. 17. The sheath 81 has a bending tendency to bend in a predetermined direction. The sheath 81 has a linear shape in a state where the sheath 81 is inserted inside the first and second through holes and the outer sheath 3 described above in the embodiment and bends in the direction of the bending tendency when the distal end part of the insertion portion 8 projects toward the distal end side Ar1 from the distal end of the outer sheath 3.

Also, as illustrated in FIG. 17, a wire 84 that is made of a conductive material and has flexibility is additionally provided in the insertion portion 8 according to the tenth modification. The wire 84 functions as a conductive path between the control device 5 and the sheath 81.

The tenth modification described above achieves the following effect in addition to the same effects as described in the embodiment.

The insertion portion 8 according to the tenth modification bends in the direction of the bending tendency due to the sheath 81 having the bending tendency when the distal end part of the insertion portion 8 projects toward the distal end side Ar1 from the distal end of the outer sheath 3.

Thus, approachability to a body part to be treated can be improved.

### Eleventh Modification

FIGS. 18 and 19 are diagrams describing an eleventh modification of the embodiment. Specifically, FIG. 18 is a perspective view of an end part of the insertion portion 8 on the distal end side Ar1 according to the eleventh modification. Note that, for convenience of description, the outer tube TO is indicated by a chain double-dashed line in FIG. 18. FIG. 19 corresponds to FIG. 3.

In the embodiment described above, the opening OP may be provided at a position in the eleventh modification illustrated in FIGS. 18 and 19.

Specifically, as illustrated in FIGS. 18 or 19, a side wall of the sheath 81 according to the eleventh modification, the side wall extending along the longitudinal axis, has a through hole 812 that penetrates the sheath 81 from the inside to outside thereof. The through hole 812 has an elliptical shape having a major axis extending along the longitudinal axis of the sheath 81 in plan view. The inner peripheral edge of the through hole 812 functions as the electrode 811.

Also, as illustrated in FIGS. 18 or 19, the non-blade member 82 according to the eleventh modification is fitted with an open part of the sheath 81 on the distal end side Ar1 to close the open part. An area on the distal end side Ar1 in the through hole 812 is closed by the non-blade member 82. In the through hole 812, a portion that is not closed by the non-blade member 82 functions as the opening OP. An opening plane of the opening OP intersects the longitudinal axis of the sheath 81. That is, a portion of the electrode 811 constituting the opening edge of the opening OP is provided on the suction direction side (the proximal end side Ar2) of the opening edge where the tissue piece LT is suctioned into the sheath 81 through the opening OP. More specifically, a portion of the opening edge of the opening OP located on the most proximal end side Ar2 is a portion on the major axis of the elliptical shape in the electrode 811. The distal end side Ar1 of the opening edge of the opening OP is constituted of the non-blade member 82 and has a linear shape perpendicular to the longitudinal axis of the sheath 81 as with the embodiment described above.

In the non-blade member 82, a surface that closes a part of the through hole 812 functions as the sliding surface 821 as illustrated in FIGS. 18 or 19. The sliding surface 821 is a curved surface concave toward the inside of the sheath 81 following the shape of the through hole 812. In the eleventh modification, as with the embodiment described above, the opening plane of the opening OP and the sliding surface 821 form a contiguous plane with no step.

In addition, as illustrated in FIGS. 18 or 19, the outer tube TO according to the eleventh modification has a through hole TO1 having an elliptical shape slightly larger than the through hole 812. The outer tube TO covers the outer face of the sheath 81 with the electrode 811, the through hole 812, the sliding surface 821, and the opening OP exposed to the outside through the through hole TO1.

Even when the position of the opening OP is changed as with the eleventh modification described above, the same effects as described in the embodiment are achieved.

### Twelfth Modification

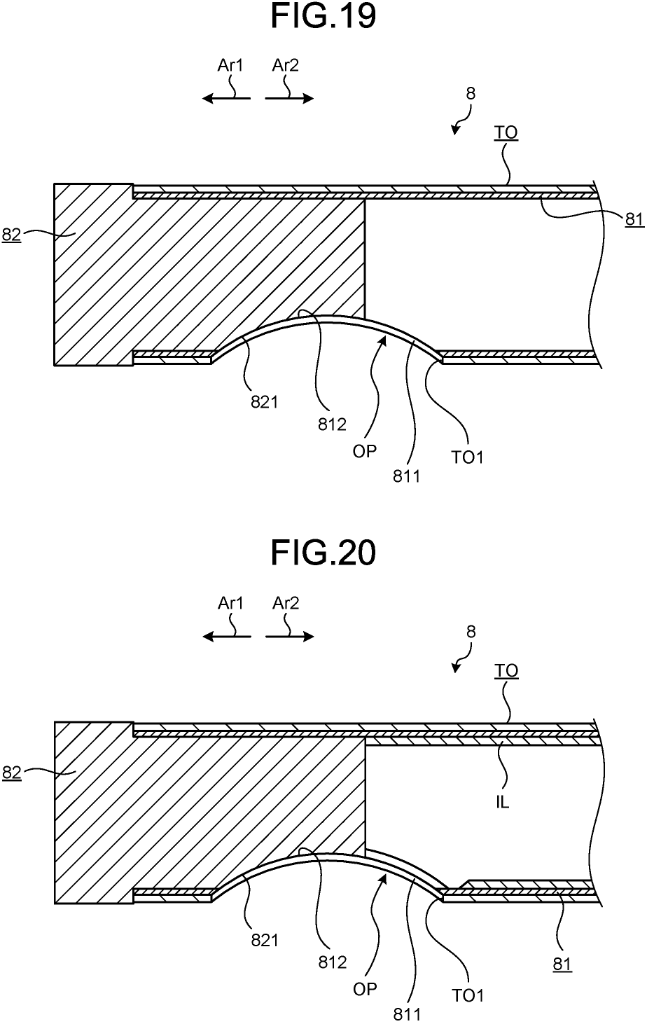

FIG. 20 is a diagram describing a twelfth modification of the embodiment. Specifically, FIG. 20 corresponds to FIG. 3.

In the eleventh modification described above, an insulating layer IL may be provided on the inner face of the sheath 81 as with the twelfth modification illustrated in FIG. 20.

The insulating layer IL is made of a material having an electrical insulating property and provided on the inner face of the sheath 81 as illustrated in FIG. 20.

The twelfth modification described above achieves the same effects as described in the embodiment and the fifth and eleventh modifications.

### Thirteenth Modification

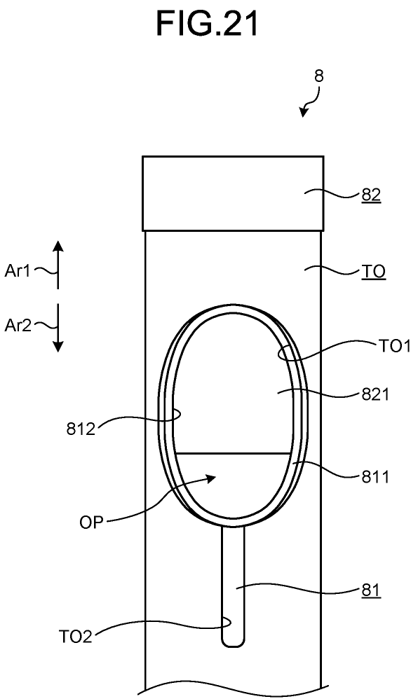

FIG. 21 is a diagram describing a thirteenth modification of the embodiment. Specifically, FIG. 21 is a diagram illustrating an end part of the insertion portion 8 on the distal end side Ar1 according to the thirteenth modification viewed in the direction perpendicular to the longitudinal axis of the insertion portion 8.

In the eleventh modification described above, the outer tube TO may be provided with a groove TO2 as with the thirteenth modification illustrated in FIG. 21.

As illustrated in FIG. 21, the groove TO2 penetrates the outer tube TO from the inside to outside thereof and linearly extends by a predetermined dimension toward the proximal end side Ar2 from a part of the inner peripheral edge of the through hole TO1 located on the most proximal end side Ar2.

A part of the outer face other sheath 81 other than the electrode 811 is also exposed through the groove TO2.

The thirteenth modification described above achieves the same effects as described in the embodiment and the fifth and eleventh modifications.

### Fourteenth Modification

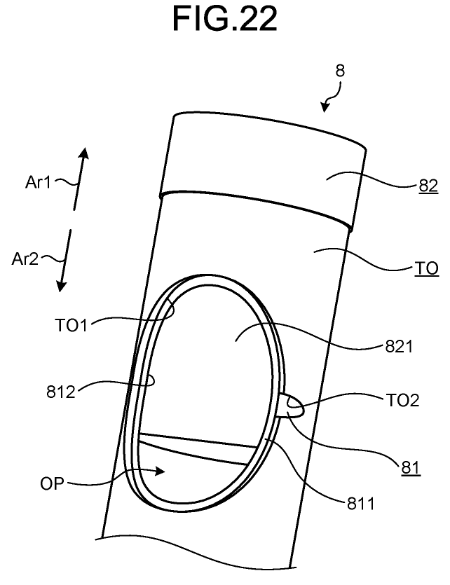

FIG. 22 is a diagram describing a fourteenth modification of the embodiment. Specifically, FIG. 22 is a perspective view of an end part of the insertion portion 8 on the distal end side Ar1 according to the fourteenth modification.

In the thirteenth modification described above, the position where the groove TO2 is provided is not limited to the part of the inner peripheral edge of the through hole TO1 located on the most proximal end side Ar2 and may be another position on the inner peripheral edge as with the fourteenth modification illustrated in FIG. 22.

Even when the position where the groove TO2 is provided is changed as with the fourteenth modification described above, the same effects as described in the thirteenth modification are achieved.

### Fifteenth Modification

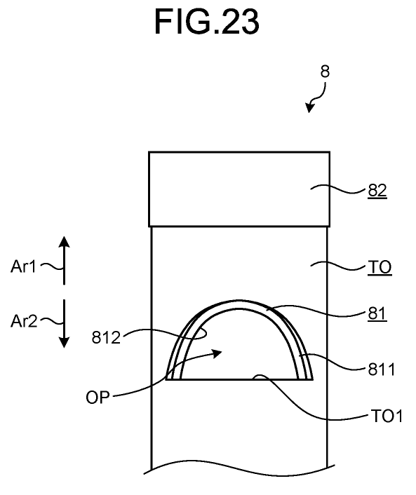

FIG. 23 is a diagram describing a fifteenth modification of the embodiment. Specifically, FIG. 23 is a diagram illustrating an end part of the insertion portion 8 on the distal end side Ar1 according to the fifteenth modification viewed in the direction perpendicular to the longitudinal axis of the insertion portion 8.

In the eleventh modification described above, the shapes of the non-blade member 82 and the outer tube TO may be shapes in the fifteenth modification illustrated in FIG. 23.

Specifically, the non-blade member 82 according to the fifteenth modification only has a function of closing an open part of the sheath 81 on the distal end side Ar1 by being fitted with the open part. That is, the non-blade member 82 does not close the through hole 812.

In addition, in the outer tube TO according to the fifteenth modification, as illustrated in FIG. 23, the through hole TO1 has a shape having only substantially half, on the distal end side Ar1, of the area of the elliptical shape of the through hole TO1 described in the eleventh modification. The outer tube TO covers the outer face of the sheath 81 with the U-shaped portion of the electrode 811 on the distal end side Ar1 and substantially half, on the distal end side Ar1, of the area of the elliptical shape of the through hole 812 exposed to the outside through the through hole TO1. The opening OP is defined by the through hole TO1 and the U-shaped electrode 811 exposed to the outside. That is, the electrode 811 is provided opposite to the suction direction side (the proximal end side Ar2) of the opening edge of the opening OP where the tissue piece LT is suctioned into the sheath 81 through the opening OP. More specifically, a portion of the opening edge of the opening OP located on the most distal end side Ar1 corresponds to a valley portion of the U shape of the electrode 811 exposed to the outside. The proximal end side Ar2 of the opening edge of the opening OP is constituted of the inner peripheral edge of the through hole TO1 and has a linear shape perpendicular to the longitudinal axis of the sheath 81.

Even when the shapes of the non-blade member 82 and the outer tube TO are changed as with the fifteenth modification described above, the same effects as described in the eleventh modification are achieved.

### Reference Signs List

1 TREATMENT SYSTEM
2 ENDOSCOPE
3 OUTER SHEATH
4 TREATMENT TOOL
5 CONTROL DEVICE
6 FOOT SWITCH
7 SUCTION DEVICE
8 INSERTION PORTION
9 HOUSING
21 ENDOSCOPE MAIN BODY
22 DISTAL END PORTION
23 TREATMENT TOOL INSERTION PORTION
24 EYEPIECE PORTION
31 WATER FEED PORT
32 WATER FEED HOLE
81 SHEATH
82 NON-BLADE MEMBER
83 LEAD WIRE
84 WIRE
91 OPERATING UNIT
811 ELECTRODE
812 THROUGH HOLE
821 SLIDING SURFACE
822 THROUGH HOLE
823 GROOVE
831 ELECTRODE
Ar1 DISTAL END SIDE
Ar2 PROXIMAL END SIDE
BL BLADDER
IL INSULATING LAYER
LT TISSUE PIECE
OP OPENING
PL PLANE
TO OUTER TUBE
TO1 THROUGH HOLE
TO2 GROOVE
UR URETHRA

What is claimed is:

1. A treatment tool, comprising:
a tubular member including an opening and a tubular member main body that has a tubular shape, the tubular member being configured to suction a tissue piece through the opening; and
a blade member provided on an opening edge of the opening.

2. The treatment tool according to claim 1, wherein the blade member is provided on a first part of the opening edge and is configured to scrape the tissue piece,
wherein the treatment tool further comprises a non-blade member that is provided on at least a second part of the opening edge, the non-blade member configured to allow the tissue piece to slide on the non-blade member, and
wherein the second part of the opening edge is outside the first part of the opening edge.

3. The treatment tool according to claim 1, wherein the blade member has a U-shape that follows a shape of the opening edge.

4. The treatment tool according to claim 1, wherein the opening is provided on a distal end of the tubular member, and wherein an imaginary plane containing the opening is inclined from a distal end side toward a proximal end side of the tubular member.

5. The treatment tool according to claim 2, wherein the opening is provided on a distal end of the tubular member,
wherein an imaginary plane containing the opening is inclined from a distal end side toward a proximal end side of the tubular member, and
wherein the non-blade member is provided on a distal end side of the opening edge and the blade member is provided on a proximal end side of the opening edge.

6. The treatment tool according to claim 1, wherein the tubular member includes a sliding surface disposed on a distal end of the tubular member,
wherein the sliding surface is configured to allow the tissue piece to slide on the sliding surface,
wherein an imaginary plane containing the sliding surface is inclined downward from a distal end side toward a proximal end side of the sliding surface,
wherein the proximal end side of the sliding surface includes a ridge line that forms a first part of the opening edge, and
wherein a second part of the opening edge forms at least a part of the blade member.

7. The treatment tool according to claim 1, wherein the blade member is an electrode configured to scrape the tissue piece in response to a supplied high frequency power.

8. The treatment tool according to claim 1, wherein the blade member is a mechanical blade configured to mechanically scrape the tissue piece.

9. The treatment tool according to claim 2, wherein the non-blade member includes a groove configured to penetrate from an inside of the tubular member main body to an outside of the tubular member main body.

10. The treatment tool according to claim 9, wherein the groove linearly extends in a circumferential direction of the tubular member main body.

11. A treatment system, comprising:
a treatment tool configured to be inserted into a subject; and
a suction device configured to suction a tissue piece through the treatment tool,
wherein the treatment tool includes:
a tubular member including an opening and a tubular member main body that has a tubular shape, the tubular member being configured to suction the tissue piece through the opening, and
a blade member provided on an opening edge of the opening.

12. The treatment system according to claim 11, wherein the blade member is an electrode configured to scrape the tissue piece in response to a supplied high frequency power or the blade member is a mechanical blade configured to mechanically scrape the tissue piece.

13. The treatment system according to claim 11, wherein the blade member is provided on a first part of the opening edge and is configured to scrape the tissue piece,
wherein the treatment tool further comprises a non-blade member that is provided on at least a second part of the opening edge, the non-blade member configured to allow the tissue piece to slide on the non-blade member, and
wherein the second part of the opening edge is outside the first part of the opening edge.

14. The treatment system according to claim 11, wherein the tubular member includes a sliding surface disposed on a distal end of the tubular member,
wherein the sliding surface is configured to allow the tissue piece to slide on the sliding surface,
wherein an imaginary plane containing the sliding surface is inclined downward from a distal end side toward a proximal end side of the sliding surface,
wherein the proximal end side of the sliding surface includes a ridge line that forms a first part of the opening edge, and
wherein a second part of the opening edge forms at least a part of the blade member.

15. A treatment method of collecting a tissue piece, comprising:
suctioning the tissue piece through an opening included in a treatment tool;
scraping off a part of the tissue piece using a blade member provided on the opening; and
suctioning, through the opening, the part of the tissue piece that was scraped off.

16. The treatment method according to claim 15, further comprising:
after suctioning the part of the tissue piece that was scraped off, suctioning to turn the tissue piece;
scraping off a second part of the tissue piece using the blade member; and
suctioning the second part of the tissue piece that was scraped off.

17. The treatment method according to claim 15, wherein the blade member is an electrode configured to scrape the tissue piece in response to a supplied high frequency power or the blade member is a mechanical blade configured to mechanically scrape the tissue piece.

18. The treatment method according to claim 15, wherein the blade member is provided on a first part of an opening edge of the opening and is configured to scrape the tissue piece,
wherein the treatment tool further comprises a non-blade member that is provided on at least a second part of the opening edge, the non-blade member configured to allow the tissue piece to slide on the non-blade member, and
wherein the second part of the opening edge is outside the first part of the opening edge.

19. The treatment method according to claim 15, further comprising a tubular member includes a sliding surface disposed on a distal end of the tubular member,
wherein the sliding surface is configured to allow the tissue piece to slide on the sliding surface,
wherein an imaginary plane containing the sliding surface is inclined downward from a distal end side toward a proximal end side of the sliding surface,
wherein the proximal end side of the sliding surface includes a ridge line that forms a first part of an opening edge of the opening, and
wherein a second part of the opening edge forms at least a part of the blade member.
